(19) United States
(12) Patent Application Publication
(10) Pub. No.: US 2015/0321794 A1
(43) Pub. Date: Nov. 12, 2015

(54) Card Case And Syringe Case

(71) Applicant: Gerardo Molano, Orlando, FL (US)

(72) Inventor: Gerardo Molano, Orlando, FL (US)

(21) Appl. No.: 14/273,584

(22) Filed: May 9, 2014

(51) Int. Cl. B65D 25/06; B65D 81/02; B65D 55/10; A61M 5/00; B65D 25/22

(52) U.S. Cl. CPC B65D 25/06; A61M 5/002; B65D 25/22; B65D 55/10; B65D 81/02

(57) ABSTRACT

A card and syringe container system wherein a card case and a syringe case are encased within a main carrier case. The card case can be used to organize business cards and the syringe case is used to organize syringes and medicines. The card case has a unique removable face plate that can be engraved with information in case of loss. The main carrying case has a first part and a second part connected to one another via a mechanism which allows the first part to move close to or away from the second part such that the main carrying case can be in a closed or opened position. The mechanism further allows the first part to bend against the second part about 90 degrees such that the card case or the syringe case can be easily accessed.

### BACKGROUND OF THE INVENTION

1. Field of the Invention

This invention relates generally to organizing containers, and more particularly to compact containers that carry multiple cases for organizing business cards and syringes.

2. Description of Related Art

Disposable hypodermic syringes for injecting drugs of various kinds have found wide-spread use in the medical and related professions. For out-patient or home delivery of injections, it would be desirable to provide a protective hypodermic syringe case which retains one or more pre-filled hypodermic syringes with the desired medication at the prescribed dose. Business card cases have been known in the art for businessmen to exchange information. For businessmen who have diabetes and need to give themselves injections may find inconvenient to carry two separate cases one for holding business cards and another for holding syringes. Therefore, it is desirable to have one case to carry both business cards and syringes in separate compartments.

### SUMMARY OF THE INVENTION

In an exemplary embodiment of the present invention, there is disclosed a card and syringe container 1 comprising a card case 100 and a syringe case 200 both of which are encased in a main carrier case 300. The card case 100 and syringe case 200 are located inside the main carrier case 300. The card case can be used to organize business cards and the syringe case is used to organize syringes and medicines.

The main carrier case 300 comprises a first part 301 and a second part 302 which are connected and can be pulled open and pushed close relative to one another. The first part 301 once pulled away from the second part 302 can be further pressed down towards the bottom of the main carrier case 300.

The card case 100 comprises a first unit 101, and a second unit 102, which are connected and can be pulled open and pushed close relative to one another. The first unit 101 once pulled away from the second unit 102 can be further pressed down towards the bottom of the card case 100.

Therein is located a face plate that represents the upper portion of the device itself. Said unique removable face plate is placed on the top of the card case which has a first unit and a second unit connected to one another. The unique removable face plate has a horizontal bar to engage a rectangular hollow space of the second unit to secure the removable face plate to the connected first and second units. On the faceplate itself a user can engrave their own information so that even if the case is lost while in the locked position identification can still be made as to the rightful owner of said device and said contents stored therein.

The syringe case 200 comprises a top unit 201 and a bottom unit 202 which are detachably attached to one another.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a side view of a card and syringe container according to an embodiment of the present invention in an opened position exposing a card case and syringe case held in a main carrier case.

FIG. 2 is a side view of the card and syringe container of FIG. 1 in an opened and bent position allowing easy access to the card case and syringe case.

FIG. 3 is a top view of the card and syringe container of FIG. 1 in an opened position exposing the card case which is on top of the syringe case.

FIG. 4 is a top view of the card and syringe container of FIG. 1 in an opened and bent position allowing easy access to the card case and syringe case.

FIG. 5 is an isometric view of a main carrier case in a closed position according to an embodiment of the present invention.

FIG. 6 is a right side view of the main carrier case of FIG. 5 in a closed position.

FIG. 7 is an exploded view of the main carrier case of FIG. 5 showing a first part and a second part.

FIG. 8 is a right side view of the main carrier case of FIG. 5 in a closed position showing the first part in the solid line and the location of the second part in a dotted line.

FIG. 9 is a sectional view of the main carrier case of FIG. 5 wherein the first part is shown as a solid line and the location of the second part is shown as a dotted line.

FIG. 10 is a sectional view along line 10-10 of the main carrier case of FIG. 5 showing the H-shaped divider separates the container into top and bottom compartments.

FIG. 11 is a top view of the main carrier case of FIG. 5 in a closed position showing the H-shaped divider.

FIG. 12 is a sectional view along line 12-12 of the main carrier case of FIG. 5 showing that the horizontal bar at the very front of the second part engages the front wall of the first part and thus stabilizes the first part and prevents the first part to move further.

FIG. 13 is a bottom view of the main carrier case of FIG. 5 in a closed position.

FIG. 14 is a top view of the main carrier case of FIG. 5 in an opened position wherein the first part is pulled up and away from the second part.

FIG. 15 is a top view of the card and syringe container of FIG. 5 in an opened position wherein the first part is pulled up and pushed down towards the bottom of the container.

FIG. 16 is a side view of the main carrier case of FIG. 5 in an opened position wherein the first part is pulled up and away from the second part.

FIG. 17 is a side view of the main carrier case of FIG. 5 in an opened position wherein the first part is pulled up and pushed back towards the bottom of the container.

FIG. 18 is a bottom view of the main carrier case of FIG. 5 in an opened position wherein the first part is pulled up and away from the second part.

FIG. 19 is a bottom view of the main carrier case of FIG. 5 in an opened position wherein the first part is pulled up and pushed down towards the bottom of the container.

FIG. 20 is an isometric view of a card case of the present invention where a removable face plate is placed on the top of the card case which has a first unit and a second unit connected to one another.

FIG. 21 is a side view of the card case showing a first unit and a second unit connected to one another of FIG. 20.

FIG. 22 is an exploded view of the card case of FIG. 20 showing the removable face plate and the insides of the first unit and second unit.

FIG. 23 is a partial sectional view of the card case of FIG. 20.

FIG. 24 is a partial sectional view of the card case of FIG. 20.

FIG. 25 is a partial top view of the card case of FIG. 20.

FIG. 26 is a partial sectional view of the card case of FIG. 20.

FIG. 27 is a partial bottom view of the card case of FIG. 20 showing the front part of the bottom where the lock and lock pin is located.

FIG. 28 is a bottom view of the removable face plate of FIG. 20.

FIG. 29A, FIG. 29B, and FIG. 29C are side views of the card case of FIG. 20 wherein the first unit and second unit are closed to one another in FIG. 29A, the first unit is pulled up in FIG. 29B, and the first unit is pulled up and bent towards the bottom of the second unit in FIG. 29C.

FIG. 30 is a bottom view of the card case of FIG. 20 wherein the first unit and second unit are closed to one another.

FIG. 31 is a top view of the card case of FIG. 20 wherein the removable face plate is removed and the first unit is pulled up.

FIG. 32 is a bottom view of the card case of FIG. 20 wherein the first unit is pulled from the second unit. Therein is seen a unique face plate.

FIG. 33 is an isometric view of a syringe case according an embodiment of the present invention wherein the syringe case is closed.

FIG. 34 is an exploded view of the syringe case of FIG. 33 wherein the top unit is separate from the bottom unit showing the inside of the bottom unit.

FIG. 35 is a front view along line 35 of the syringe case of FIG. 33.

FIG. 36 is a sectional view along line 36 of the syringe case of FIG. 33.

FIG. 37 is a back view along line 37 of the syringe case of FIG. 33.

FIG. 38 is a bottom view of the syringe case top cover.

### DESCRIPTION OF THE PREFERRED EMBODIMENT

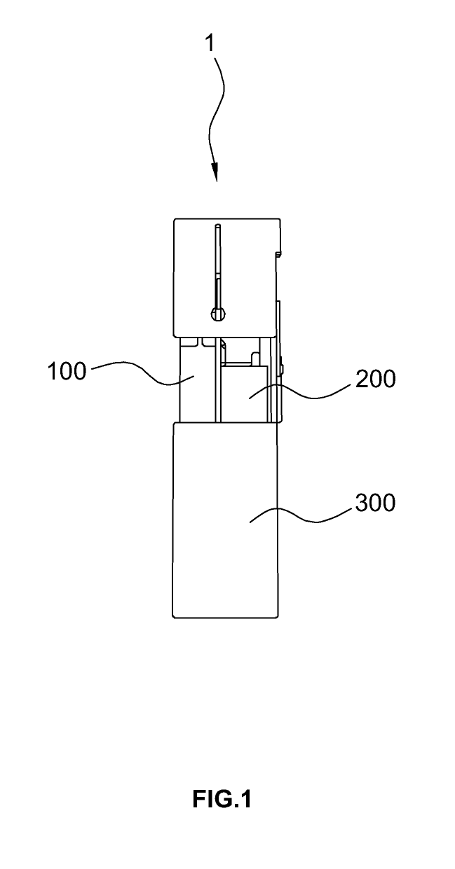

Referring to FIGS. 1-4 there is disclosed a card and syringe container 1 comprising a card case 100 and a syringe case 200 both of which are encased in a main carrier case 300. The card case 100 is located on top of the syringe case 200 inside the main carrier case 300.

FIG. 1 is a side view of a card and syringe container 1 according to an embodiment of the present invention in an opened position exposing a card case 100 and syringe case 200 held in a main carrier case 300.

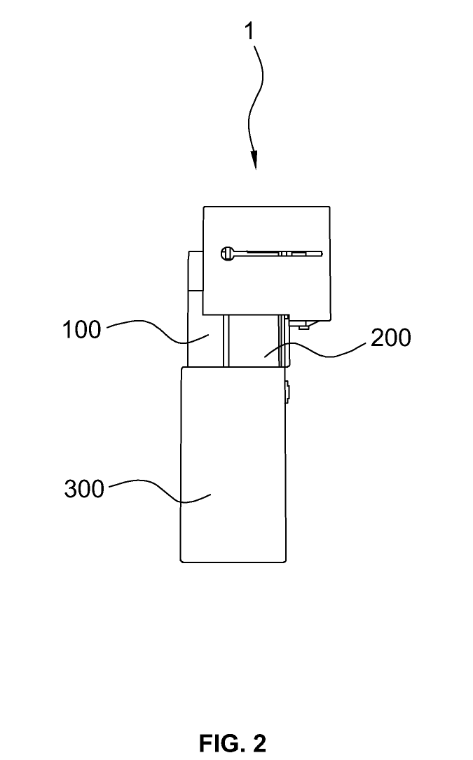

FIG. 2 is a side view of the card and syringe container 1 of FIG. 1 in an opened and bent position allowing easy access to the card case 100 and syringe case 200.

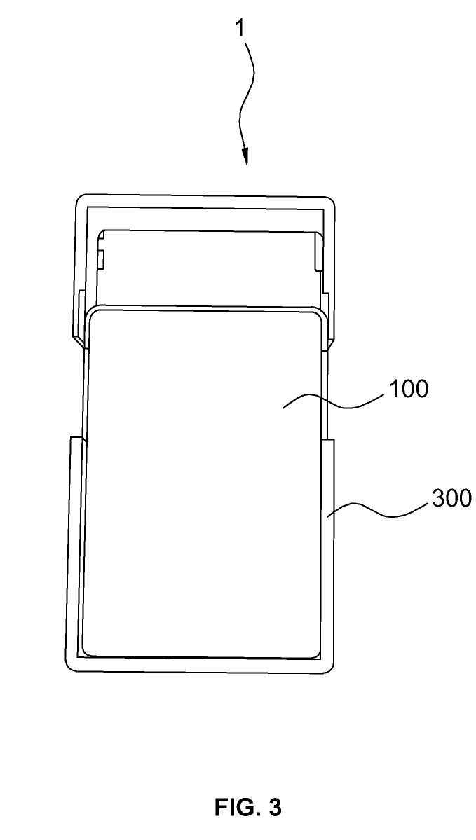

FIG. 3 is a top view of the card and syringe container 1 of FIG. 1 in an opened position exposing the card case 100 which is on top of the syringe case which is not shown in this figure.

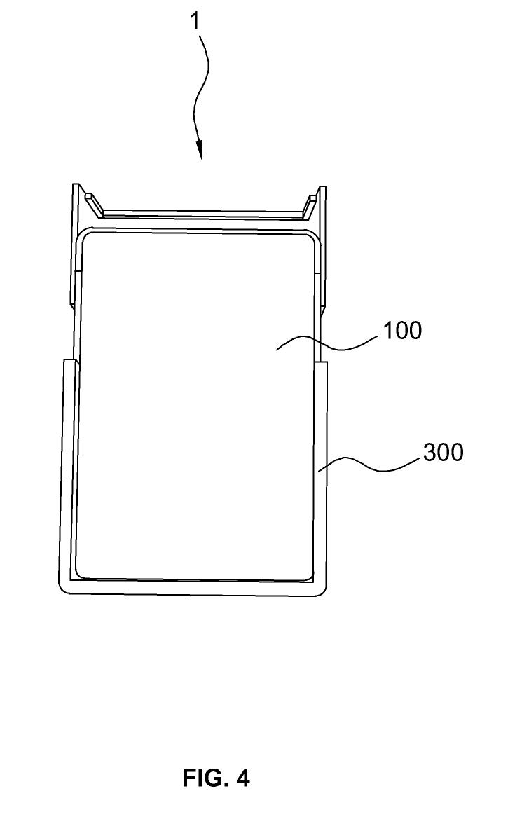

FIG. 4 is a top view of the card and syringe container 1 of FIG. 1 in an opened and bent position allowing easy access to the card case 100 and syringe case which is not observable in this figure.

Card and Syringe Main Carrier Case 300

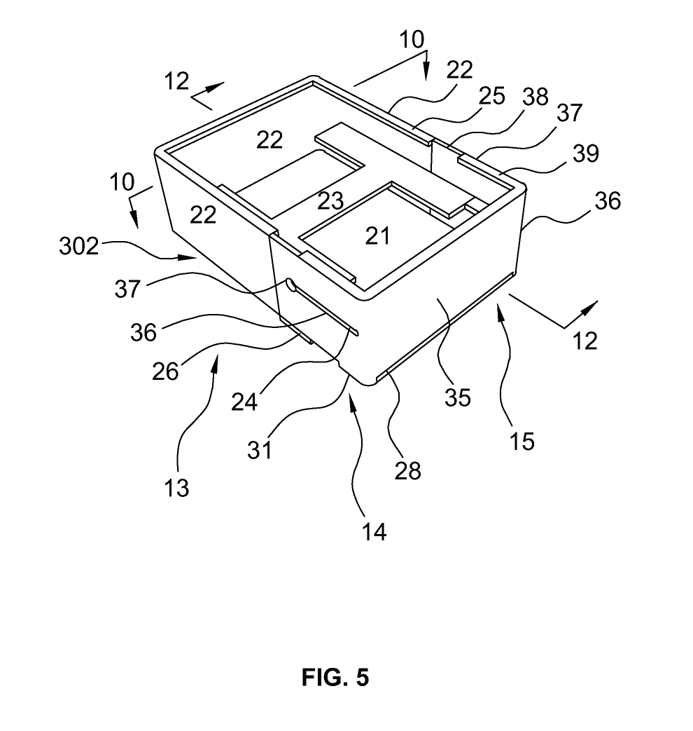

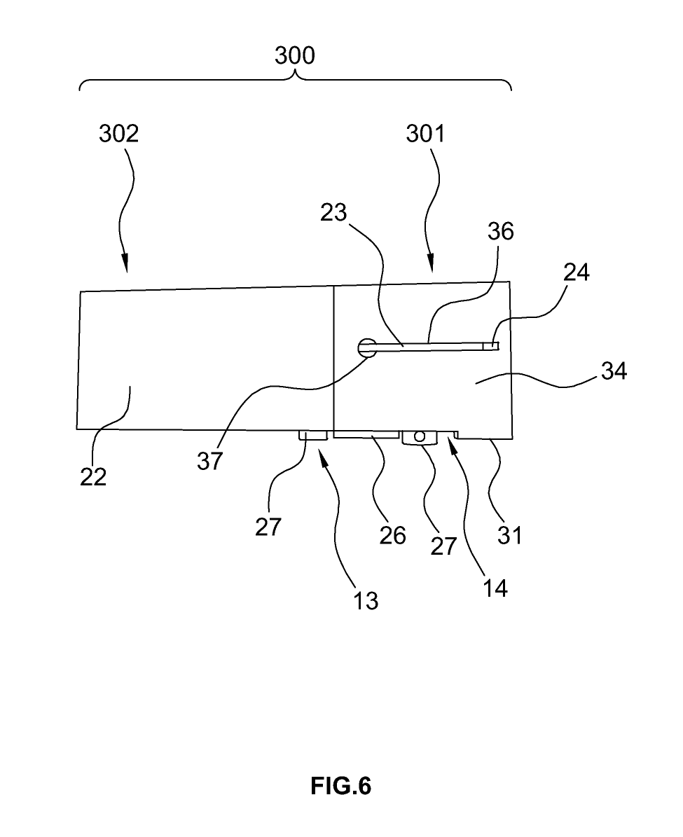

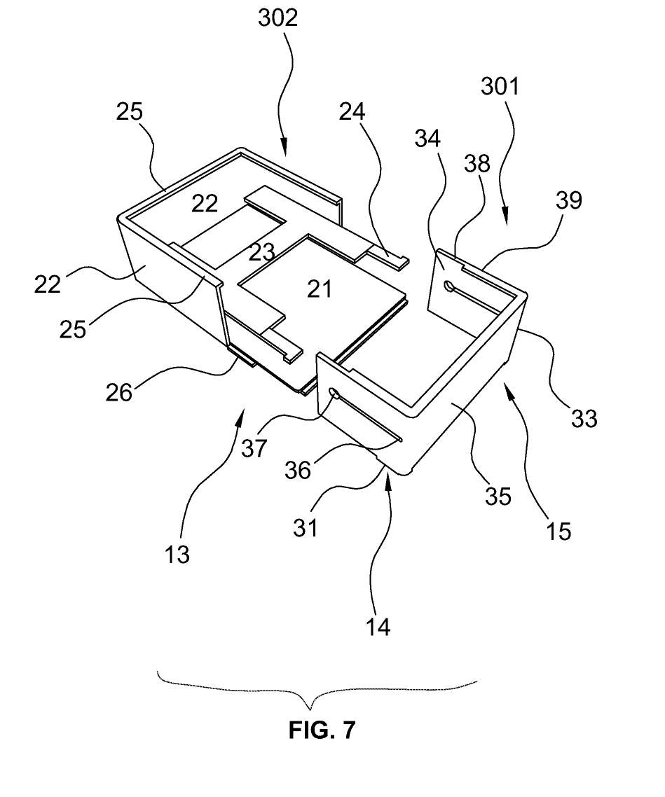

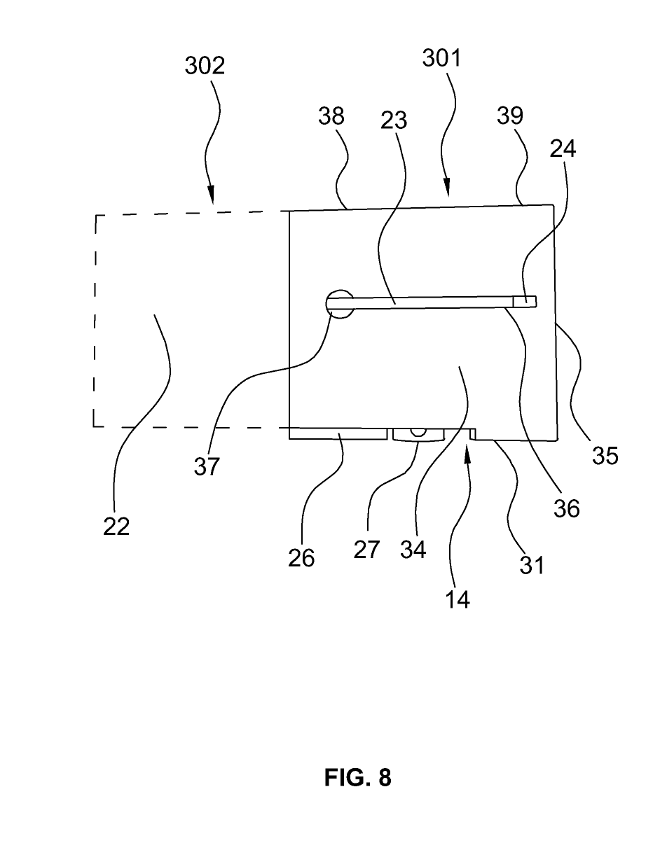

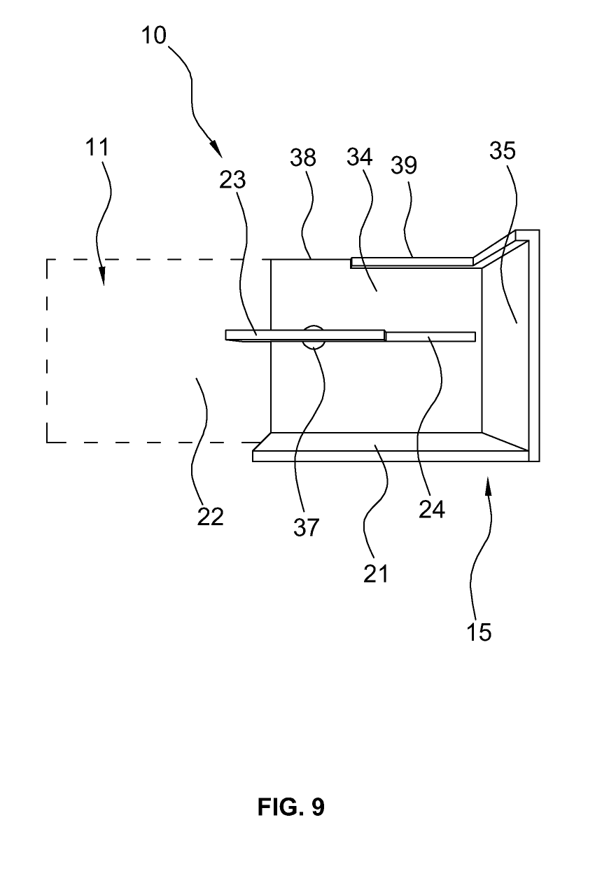

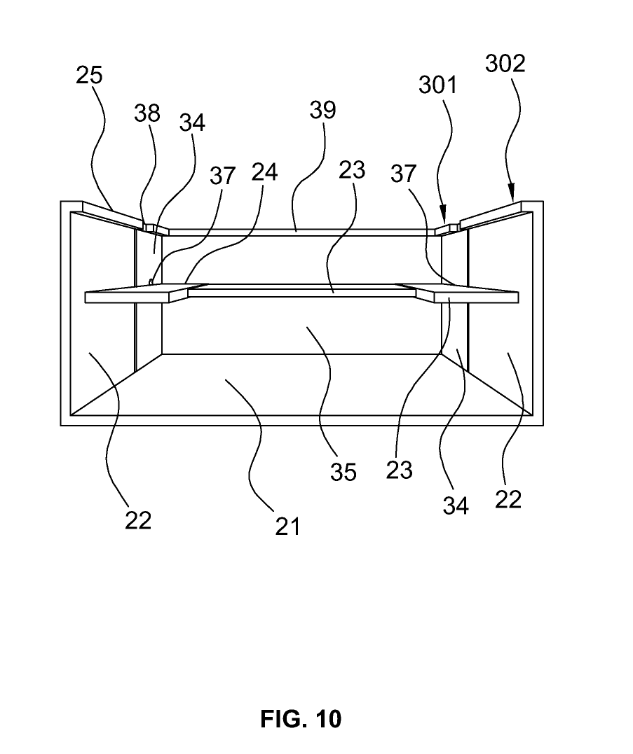

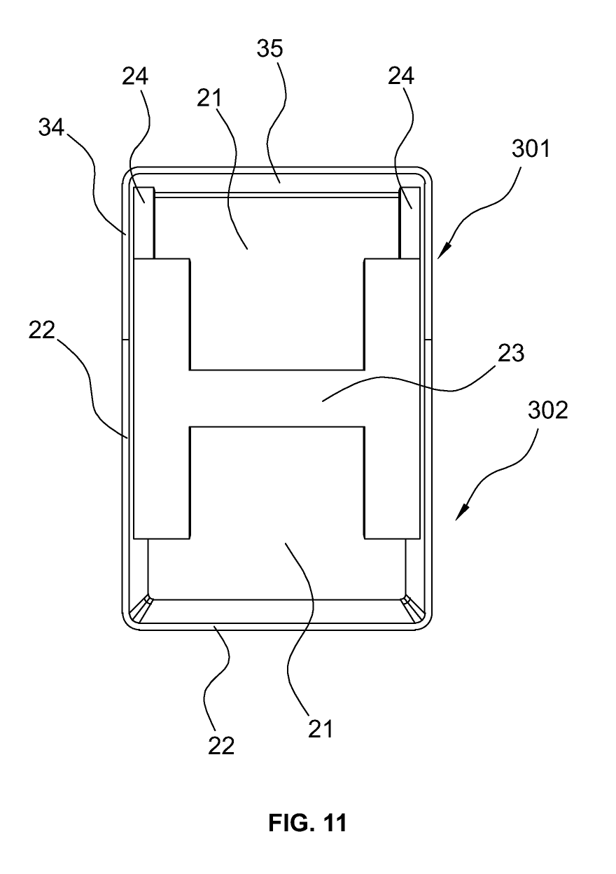

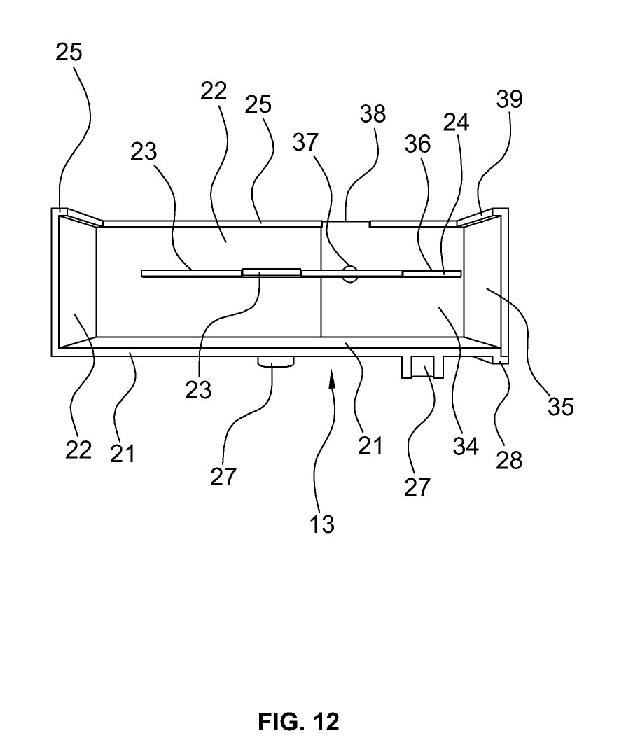

Referring to FIGS. 5-19 there is disclosed a main carrier case 300 according to the present invention. The main carrier case 300 comprises a first part 301 and a second part 302. The first part 301 and second part 302 are connected and can be pulled open and pushed close relative to one another. The first part 301 once pulled away from the second part 302 (while still remained connected) can be further pressed down towards the bottom of the main carrier case 300.

The second part 302 comprises a bottom wall 21, and several side walls 22 including a left, right, and back side walls, all of which have an inside and outside. Flange 25 perpendicularly projects from the top of the side walls towards inside of the container to prevent the card case (not shown) from falling off the main carrier case 300. The second part 302 further comprises an H-shaped divider 23 that is parallel to the bottom wall 21 and is connected to the inside of the left and right side walls 22. The H-shaped divider 23 separates the space inside the main carrying case 300 into two compartments to separately keep the card case and syringe case. The H-shaped divider 23 supports the card case 100, separating the card case 100 from the syringe case 200. At the front edge of both H-shaped divider 23 located an L-shaped wing tips 24 that protrude out to the left and to the right side of the first part 301. The L-shaped wing tips 24 are connected through circular holes 37 on the first part 301; together they hold the first part 301 and second part 302 together and able to spin the first part 90 degrees towards the bottom. Also L-shaped wing tips 24 guide the first part to move back and forth when connected to long rectangular strip openings 36. The main carrier case 300 further comprises rectangular bars 26 which locate at the mid left and mid right side of the bottom wall 21. The rectangular bars 26 prevent the first part 301 from moving when closed and connected with the second part 302. The second part 302 further comprises a long horizontal rectangular bar 28 extending from the front of the bottom wall 21; the bar 28 engages the front wall 35 of the first part 301 and thus stabilizes and prevents the first part 301 from moving while closed and attached with the second part 302.

The first part 301 comprises a front wall 35 and left and right side walls 34. All the walls have insides and outsides. The front wall 35 engages the bar 28 of the second part 302 and thus prevents the first part 301 from moving while closed and attached with the second part 302. The right and left side walls 34 engage both the left and right bars 26 of the second part 302 and thus stabilize and prevent the first part 301 from moving while closed and attached with the second part 302. The first part 301 may further comprise a rectangular rounded corner 31 located at the bottom front right corner of the first part 301 which prevents the first part 301 from moving.

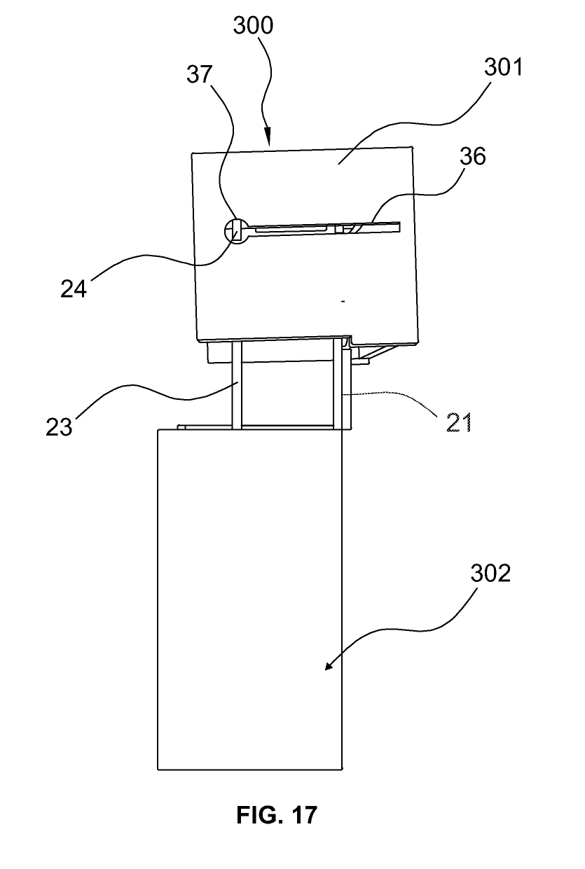

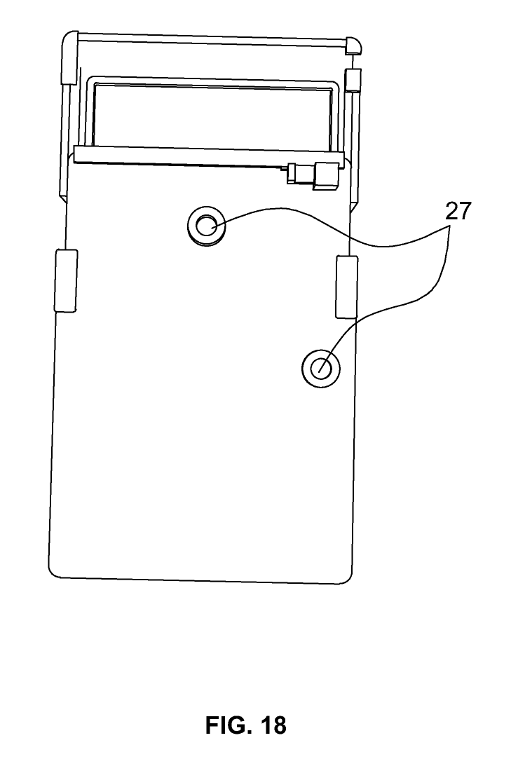

Flange 39 perpendicularly projects from the top of the front wall 35, partial right and left side walls 34 towards the inside of the container to prevent the card case (not shown) from falling off the main carrier case 300. The portion of the left and right side walls 34 that has no flange 38, forms narrow openings (squared holes) located on both the left and right side walls at the rear end of the first part 301 (embodiment with or without flanges) These openings will allow the first part 301 (while pulled all the way up) to bend down for easy access of card case, and syringe case (not shown). The first part 301 further comprises long rectangular strip openings 36 located at the mid left and the mid right sides 34 of the first part 301. These strips while engaged to the L-shaped wing tips 24 will allow and guide the first part 301 to move back and forth. The first part 301 further comprises circular holes 37 connected to the rear end of the long rectangular strip openings 36. When engaged and combined with L-shaped wing tips 24 these circular holes 37 will allow the first part 301 to move 90 degrees towards the bottom of the container. (See FIG. 17 page 11)

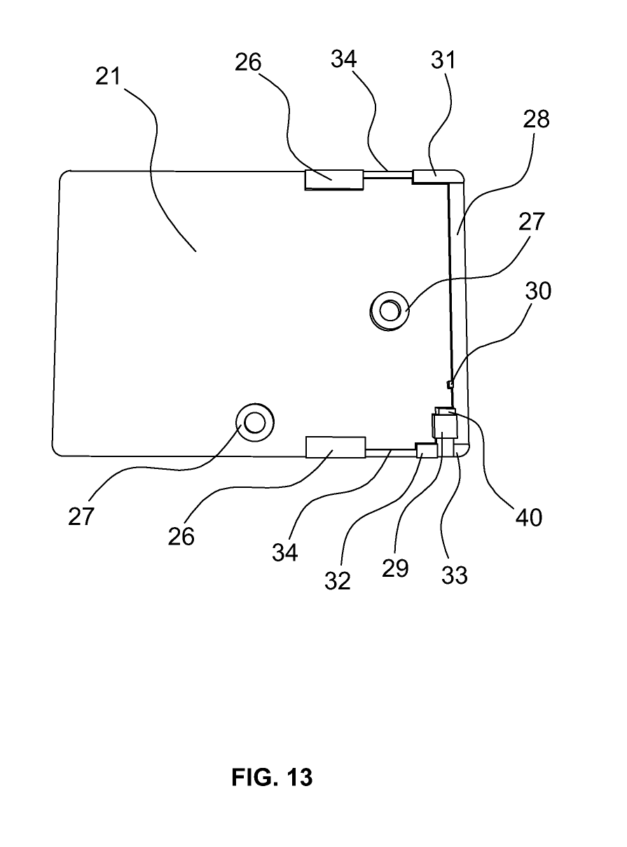

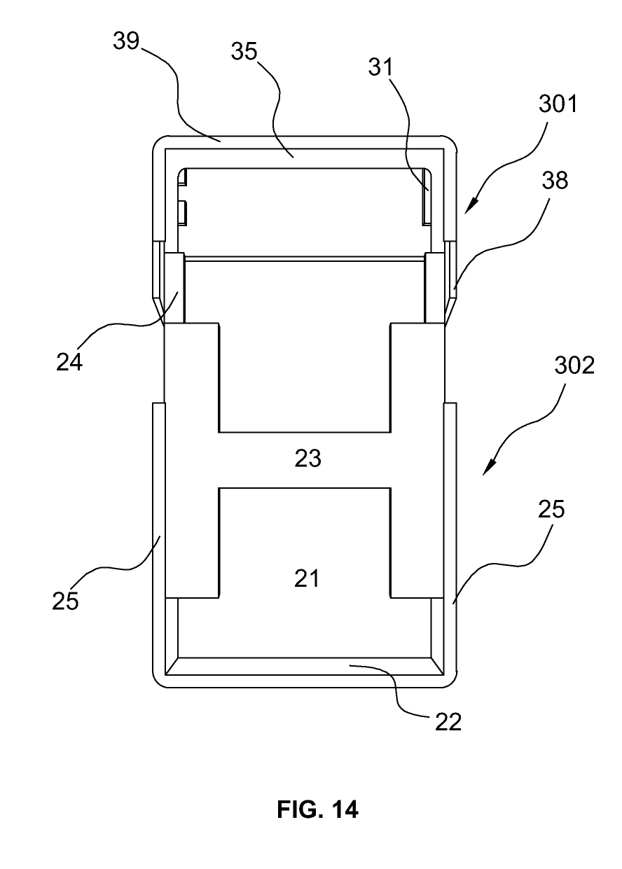

Referring to FIG. 13 there is disclosed a bottom view of the card and syringe main carrier case 300. The second part 302 further comprises a lock pin 40 located on the outside of the bottom wall 21 which will lock the parts together when pushed towards the left side of the case. It will also unlock and open the case when this pin is pushed towards the right side of the case. A rectangular part 32 located at the bottom front left corner of the first part prevents the first part 301 from moving while closed locks the lock pin 40 when inserted. A triangular shaped part 33 located at the bottom front left corner of the first part 301 stabilizes the first part 301 and helps the lock pin 40 secured in its place. Also, a rectangular rounded corner 31 located at the bottom front right corner of the first part 301 prevents the first part 301 from moving.

The second part 302 (embodiment with or without flanges) further comprises a round circular outlets 27 for the clip (not shown) to hook on to the bottom of the container horizontally or diagonally. A squared bar 29 with a rectangular hole inside is located at the very front left of the second part 302, the bar 29 holds the lock pin 40 when the container is locked or opened. A mini square tip 30 located at the left side of the bottom part 302 to prevent the lock pin 40 from falling off the unit.

FIG. 14 is a top view of the card and syringe container of FIG. 5 in an opened position wherein the first part is pulled up and away from the second part.

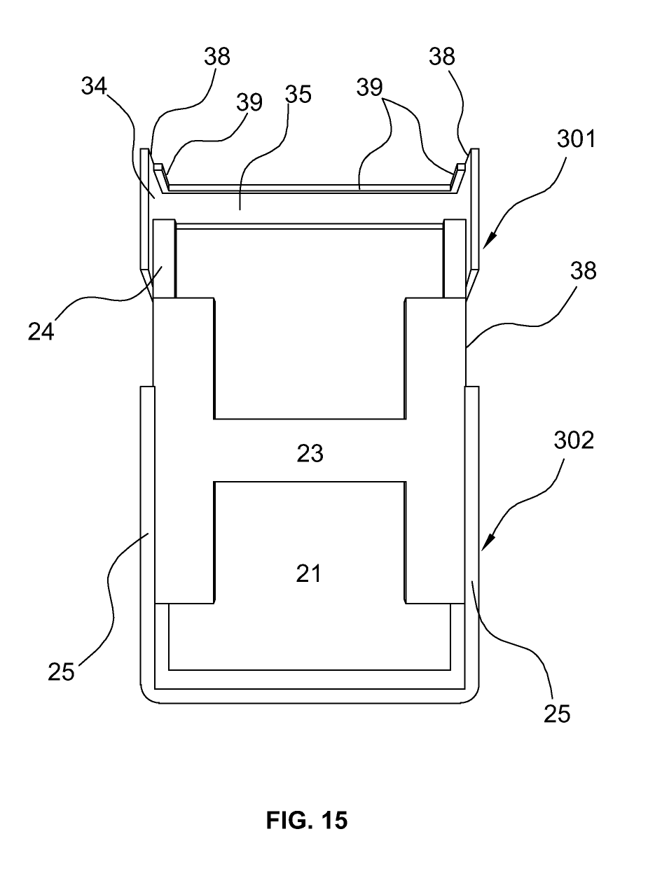

FIG. 15 is a top view of the card and syringe container of FIG. 5 in an opened position wherein the first part is pulled up and pushed down towards the bottom of the container.

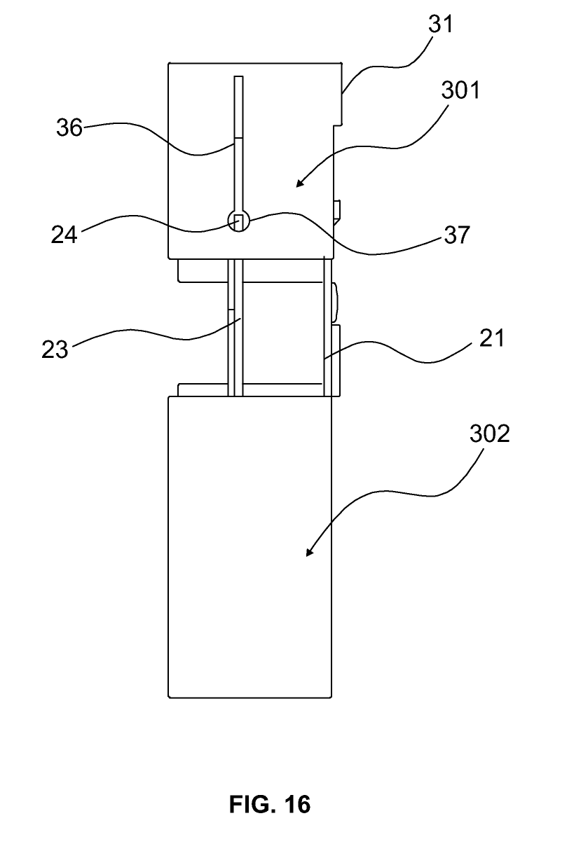

FIG. 16 is a side view of the card and syringe container of FIG. 5 in an opened position wherein the first part is pulled up and away from the second part.

FIG. 17 is a side view of the card and syringe container of FIG. 5 in an opened position wherein the first part is pulled up and pushed back towards the bottom of the container.

FIG. 18 is a bottom view of the card and syringe container of FIG. 5 in an opened position wherein the first part is pulled up and away from the second part.

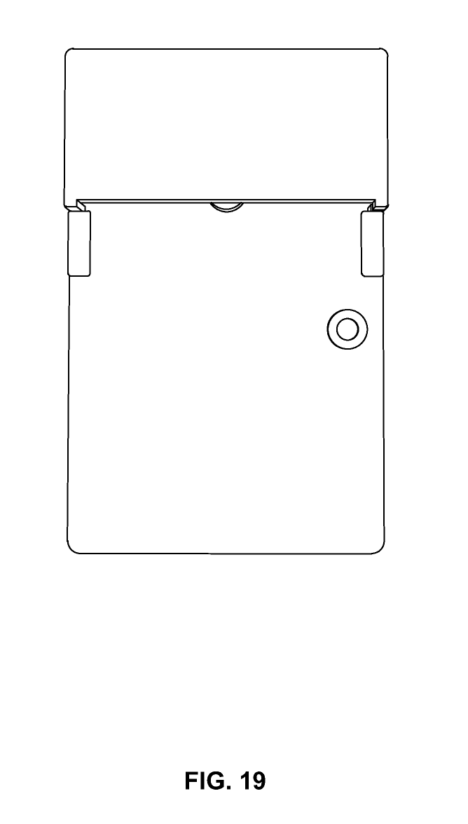

FIG. 19 is a bottom view of the card and syringe container of FIG. 5 in an opened position wherein the first part is pulled up and pushed down towards the bottom of the container.

Card Case

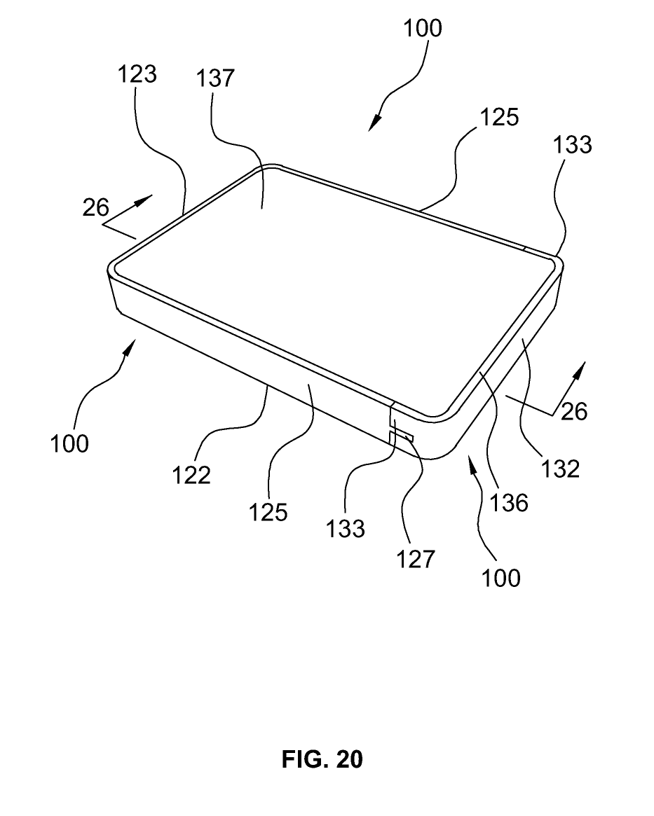

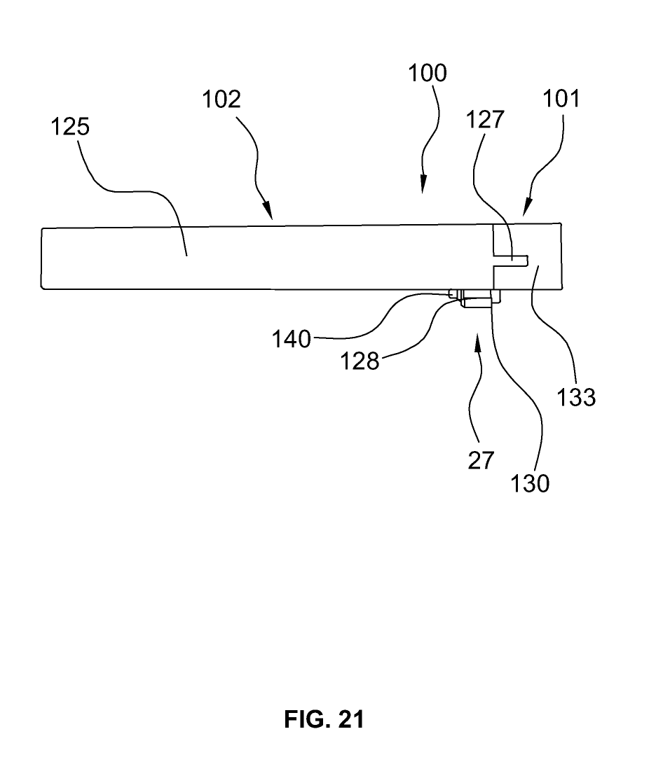

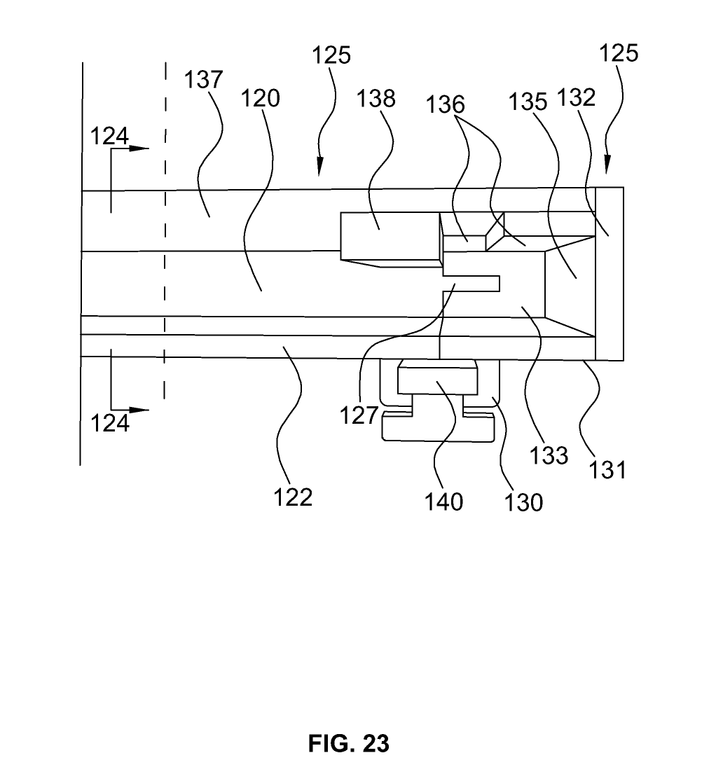

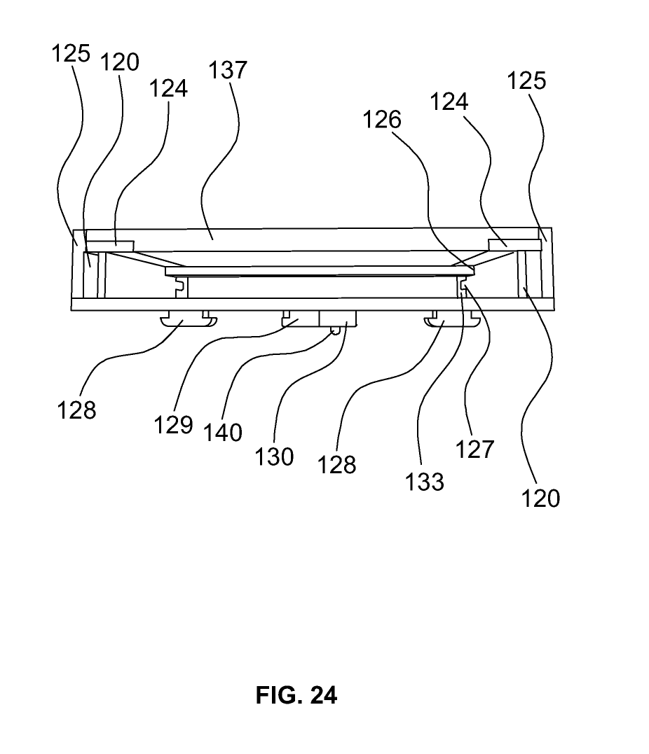

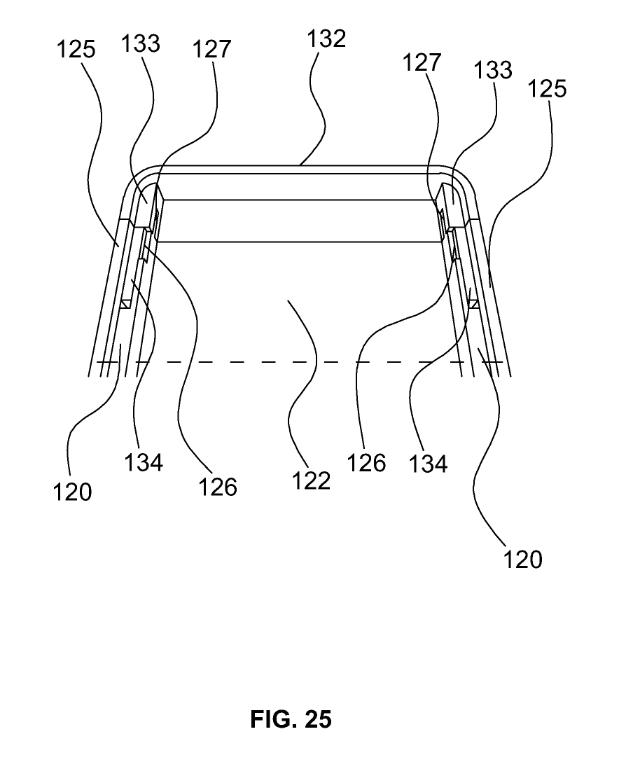

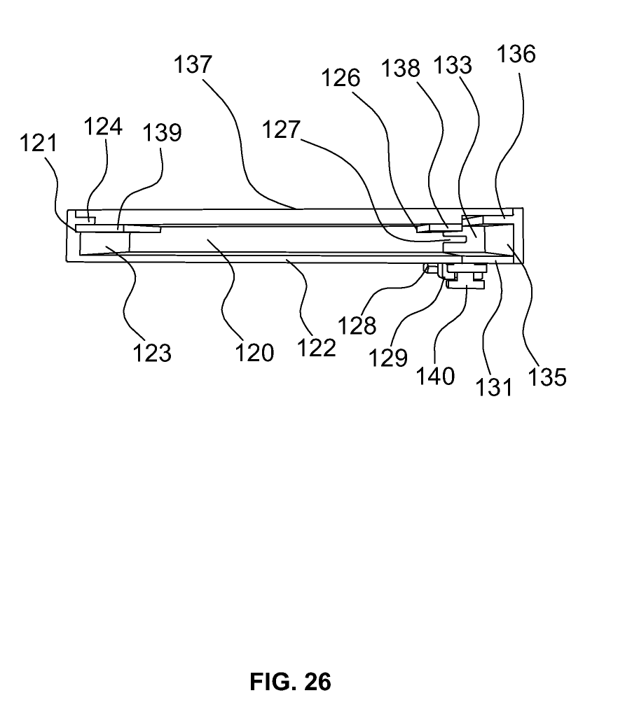

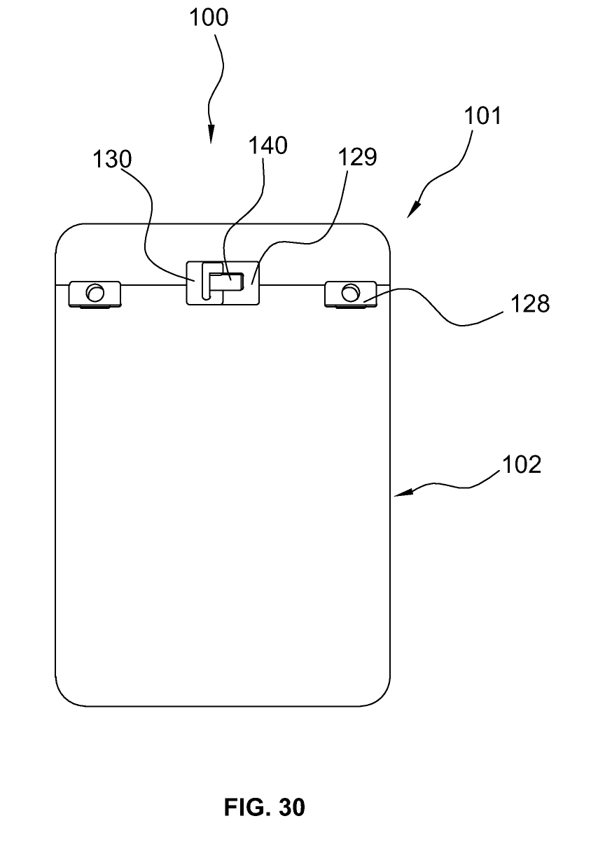

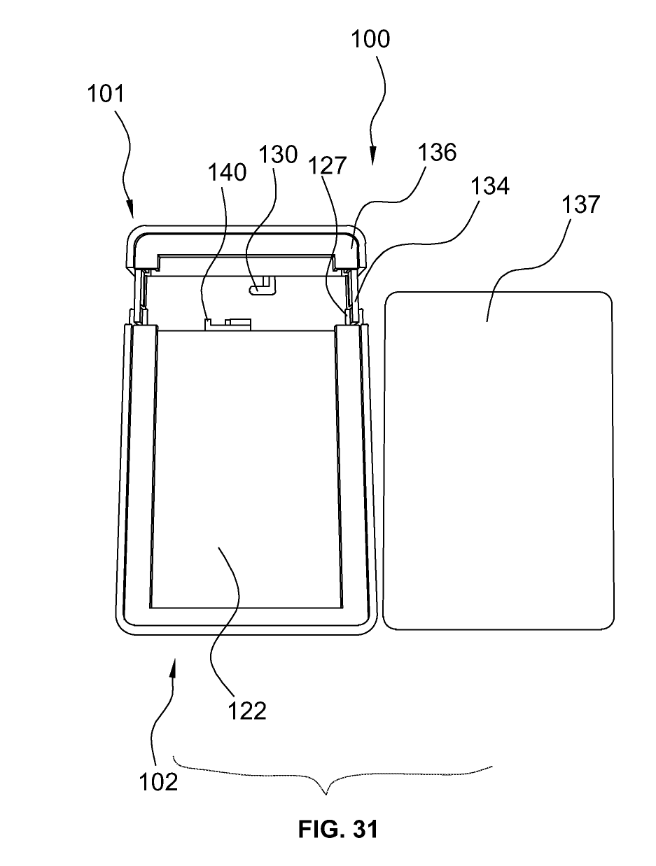

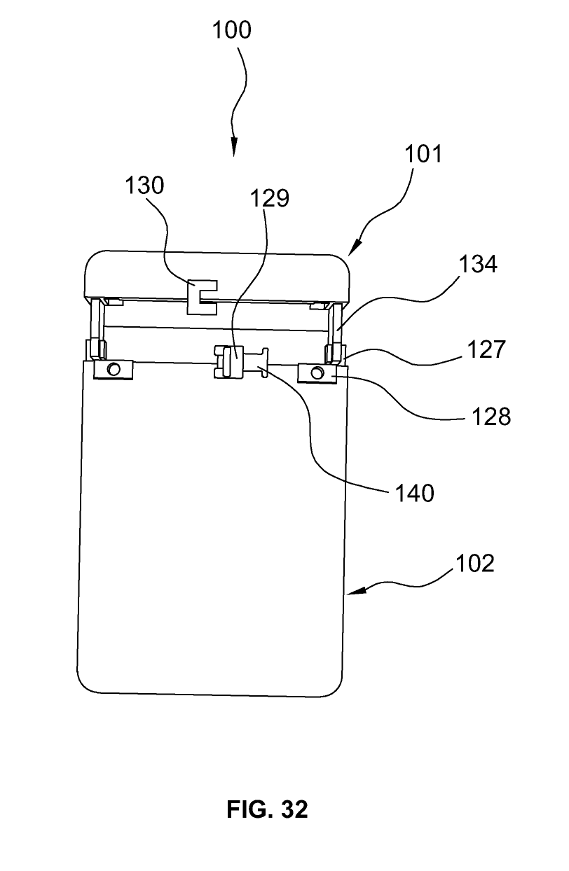

Referring to FIGS. 20-32 there is disclosed a card case 100 according to the present invention. The card case 100 comprises a first unit 101, and a second unit 102. The first unit 101 and second unit 102 are connected and can be pulled open and pushed close relative to one another. The first unit 101 once pulled away from the second unit 102 can be further pressed down towards the bottom of the card case 100.

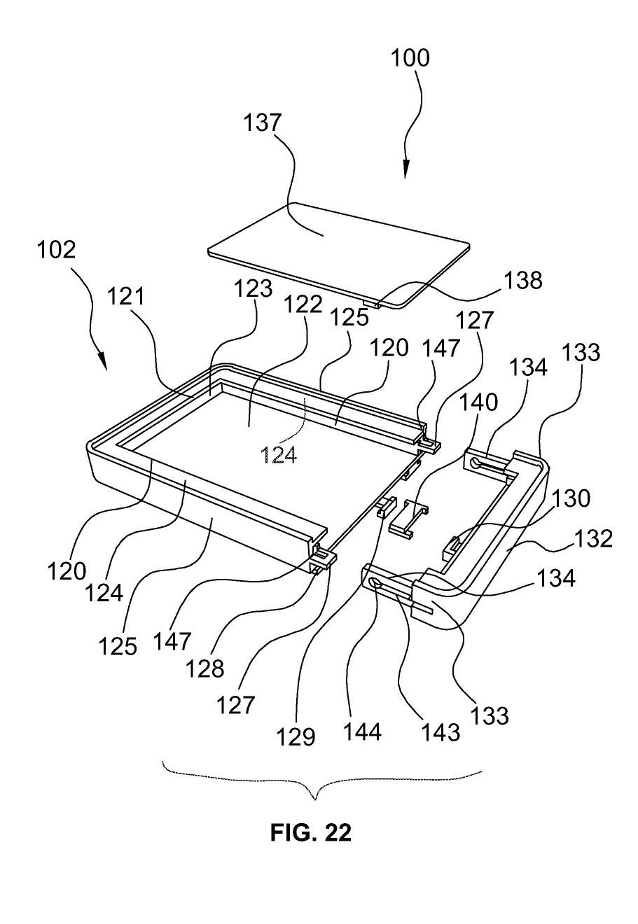

The second unit comprises a bottom wall 122, left and right inside walls 120, left and right outside walls 125, back wall 123, and long strip open space 121 located at the inside of back wall. The space 121 allows a long rectangular leg insert 139 located at the bottom of 137 to insert into. Flange 124 projects perpendicularly from the top of the side walls towards inside of the card case. The second unit 102 further comprises two U-shaped bars 127 connected to the insides 120 and outsides 125 of left and right walls. Each of the U-shaped bars 127 has a hole 147 in the middle (see particularly FIG. 22).

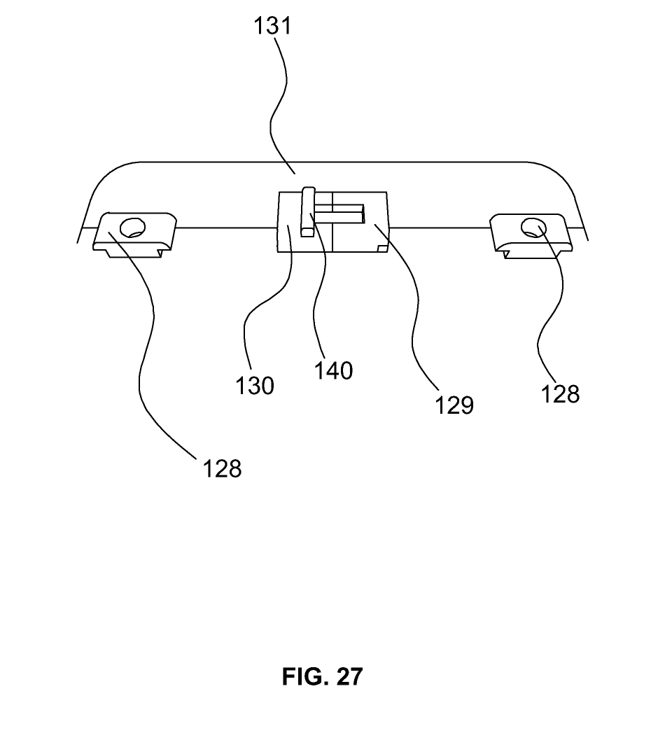

The second unit 102 further comprises rectangular hollow spaces 126 located inside left and right top corners of the second unit 102 for receiving the two horizontal bars 138 located at the bottom of the removable face plate 137 to insert and secure into. The backwards C shaped lock 129 located at the outside of the bottom wall 122 near the front of the second unit 102 has a hollow rectangular shape inside and also has squared openings at top and bottom to secure the lock pin 140 inside (see particularly FIGS. 27, 30, 32). The second unit 102 further comprises several bars 128 located at the outside of the bottom wall 122 near the front of the second unit 102; these bars 128 have screw holes in the middle for a clip (not shown) to hook onto (see particularly FIG. 27, 30, 32).

The first unit 101 of the card case comprises a front wall 132, a bottom wall 131, left and right side walls 133 connected to the front wall 132. All walls have inside and outside. The first unit 101 further comprises a small u shaped top frame 136 connected to the front wall 132, and an inside top part 135 of the first unit 101.

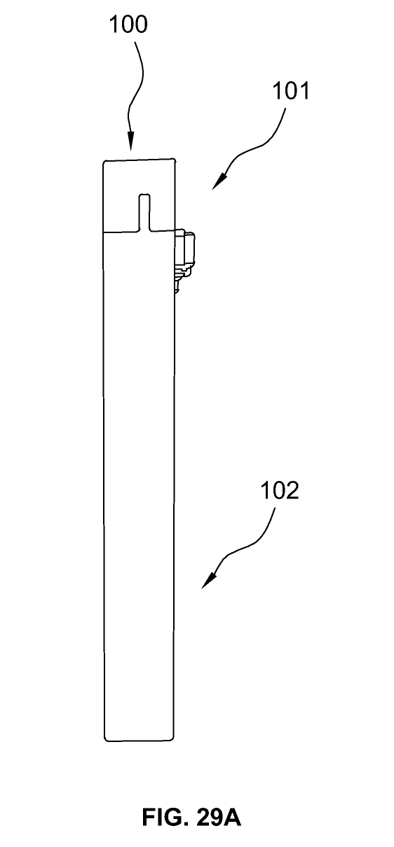

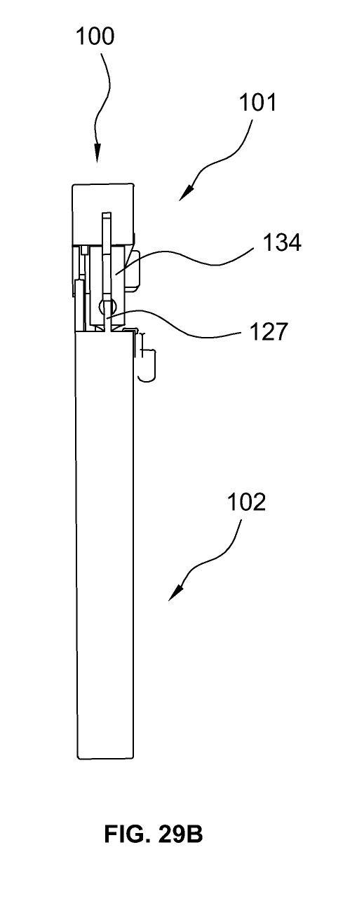

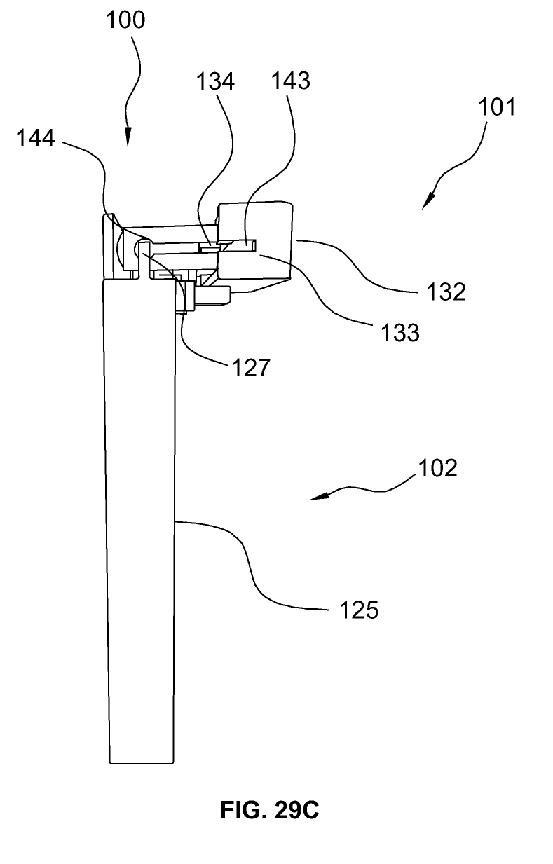

The first unit 101 further comprises two rectangular bars 134 extending from the back of the right and left side walls 133 (see particularly FIG. 22). Each of the rectangular bars 134 has a rectangular opening 143 and a circle opening 144 at the back of the rectangular opening 143. The circle openings 144 and rectangular openings 143 combined look like key holes. The left and right side bars 134 have the long rectangular openings 143 to engage the two U shaped bars 127 whereby the first and second units are attached to one another, closed, and inseparable. The bars 134 together with U shaped bars 127 prevent the units (101 and 102) from shifting to the left or the right when closed. The rectangular openings 143 in the bars 134 guide and allow the two U-shaped bars 127 to move back and forth therein, allowing the first unit 101 and second unit 102 to be pushed close (see particularly FIG. 29A) or pulled away (see particularly 29B) from one another while still remain connected via the rectangular bars 134 and U-shaped bars 127. When the first unit 101 is pulled all the way up (see particularly FIG. 29B), the circle openings 144 pulled against and touching the holes 147 of the U-shaped bars 127, the circle openings 144 allow the first unit 101 to bend approximately 90 degrees relative to the second unit 102 (see particularly FIG. 29C). When the U-shaped bars 127 combined with the rectangular bars 134, together they work like hinges.

The first unit 101 of the card case further comprises a C-shaped lock 130 located in the middle of the outside of the bottom wall 131 for receiving the lock pin 140 that is attached to the second unit 102 (see particularly FIGS. 27, 30, 32).

The first unit 101 and second unit 102 can be locked together and unlocked using the lock pin 140 when combined with the backwards C-shaped lock 129 in the second unit and the C-shaped lock 130 in the first unit.

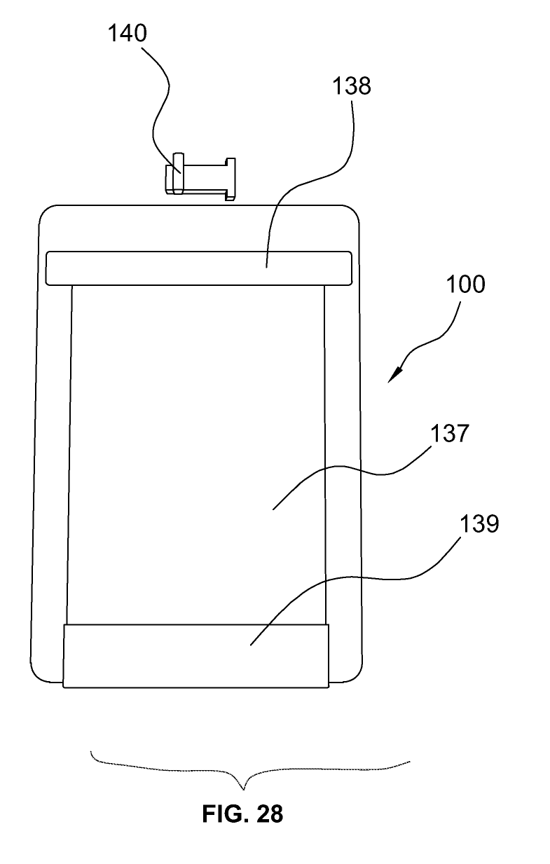

The card case 100 further comprises a removable face plate 137 that can be attached to and removed from the connected first and second units using a horizontal bar 138 located at the bottom of the removable face plate 137 (see particularly FIG. 28). The horizontal bar 138 has a left and right wing tips that can be inserted and secure into the rectangular hollow spaces 126 at the left and right top corners of the second unit 102. Also see FIG. 28, a long rectangular leg insert 139 located at the bottom of face plate 137 can be inserted one strip hollow bar that starts from 120 and ends to the other side 120 into the inside left and right side walls with rectangular hole at the top 120 to secure the face plate 137.

Syringe Case

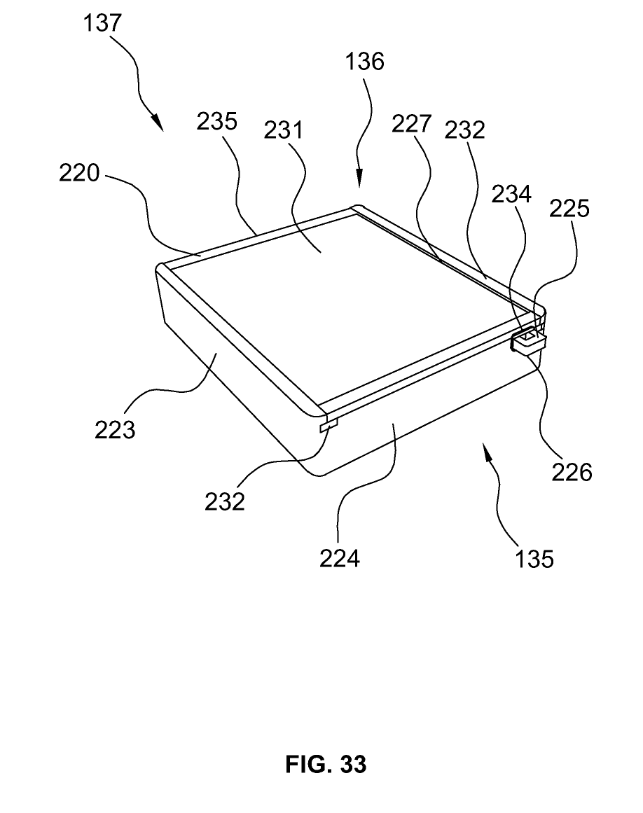

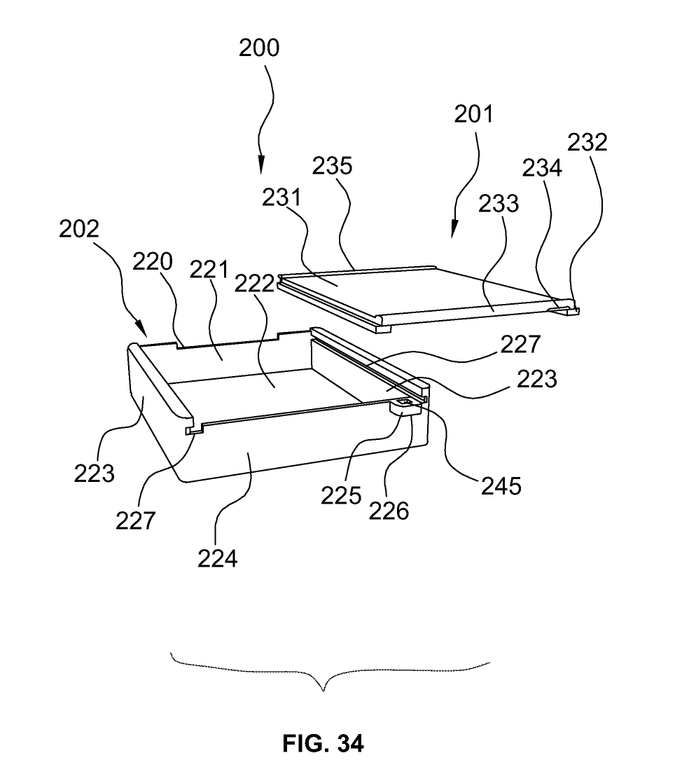

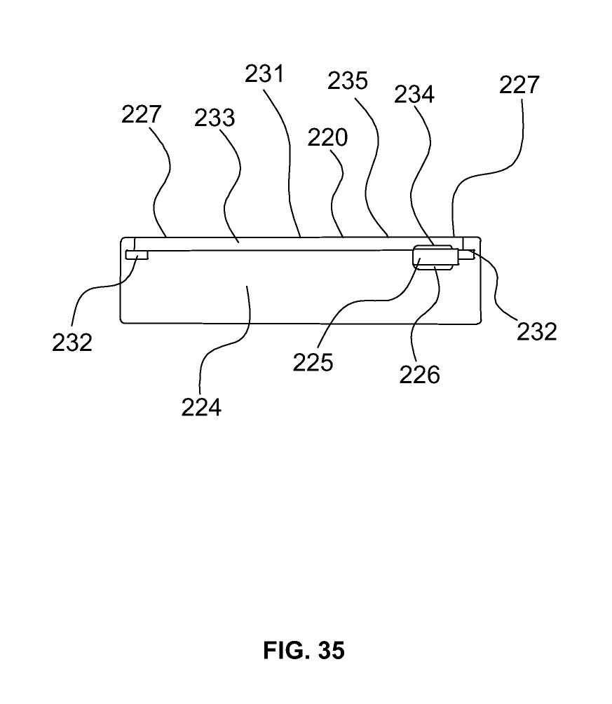

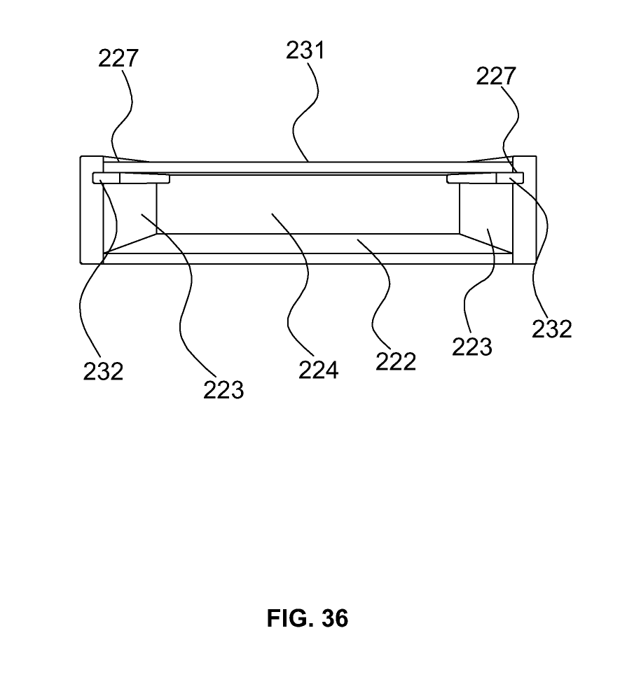

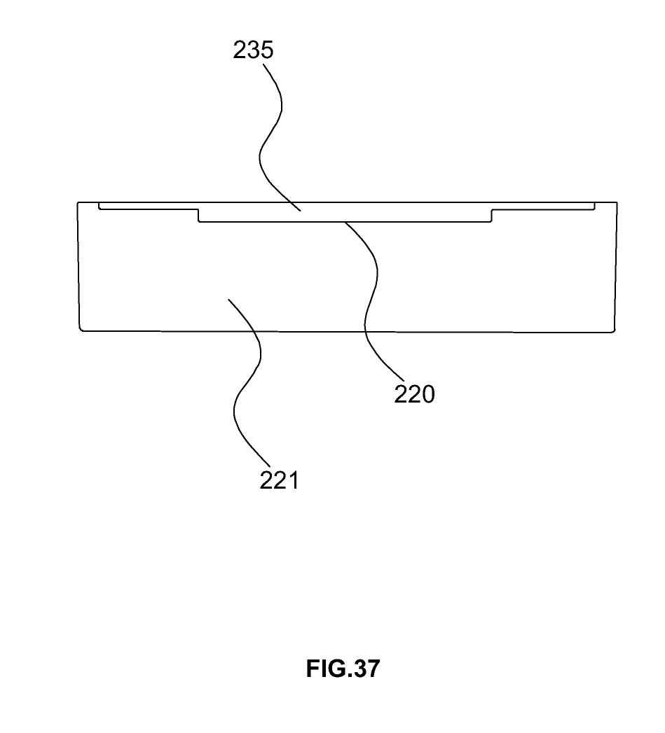

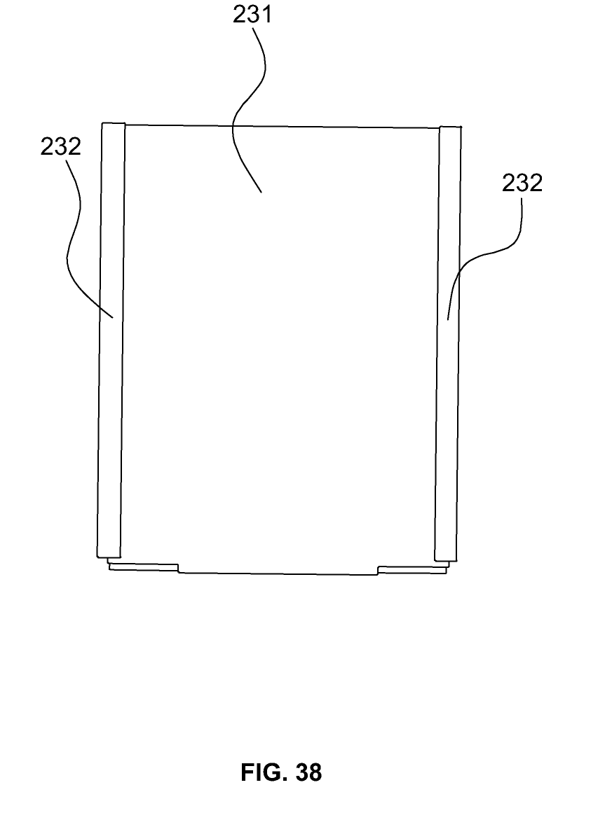

Referring to FIGS. 33-38 there is disclosed a syringe case 200 according to the present invention. The syringe case 200 comprises a top unit 201 and a bottom unit 202. The bottom unit 202 has a back wall 221, a bottom wall 222, a left and right side walls 223, and a front wall 224. All the walls have both inside and outside. Two long rectangular hollow spaces 227 located inside the right and left side walls 223 for two bars 232 of the top unit 201 to insert through, working like rails for two bars 232 to move back and forth. The back wall 221 will stop the roof 231 of the top unit 201 from sliding all the way past the back 221 when connected. A space opening 220 located at the top middle of the back wall 221 allows the back edge 235 to stick out towards the back. The syringe case 200 further comprises a square shaped bar 225 connected to the front of the front wall 224. The square shaped bar 225 has a hole 245 through the middle allowing a key ring or a small lock to go through to secure and lock the top and bottom units 201, 202 when the two combine together. A small strip bar served as a scratch guard 226 located below the square bar 225 is used to prevent the key ring or a lock from scratching the unit.

The back of the unit is flush with the side wall surface and contains a flat design. Said back section is connected to each of the four sides of the device whereby a tight seal container is formed.

The top unit 201 has a roof 231, a front edge 233, a back edge 235, and two parallel bars 232 located under the right side and left side across the front and back of the roof 231. These two bars 232 when inserted into the hollow spaces 227 close and secure the contents of the syringe case 200. The top unit 201 further comprises a scratch guard located at the right front corner of the front wall 233. When the top unit 201 is attached to the top of the bottom unit 202, this scratch guard 234 will prevent the top unit 201 from being scratched by a key ring or a lock.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A container for carrying business cards and syringes, comprising:
a card case for carrying cards;
a syringe case for carrying medications; and
a main carrying case for carrying the card case and syringe case.

2. The container of claim 1, wherein the main carrying case comprises a first part and a second part connected to one another via a mechanism which allows the first part to move close to or away from the second part such that the main carrying case is in a closed or opened position and which allows the first part to bend against the second part about 90 degrees such that the card case or the syringe case can be easily accessed.

3. The container of claim 2, wherein the second part comprises a bottom wall, several side walls including a left, right, and back side walls, and a divider that is parallel to the bottom wall and is connected to the left and right side walls for partitioning a space inside the main carrying case into two compartments to separately keep the card case and the syringe case.

4. The container of claim 3, wherein the first part comprises a front wall, right and left walls, and two long rectangular strip openings, each of the strip openings connected with a circular opening at one end, each of the strip openings separately located in the middle of the right and left walls.

5. The container of claim 4, wherein the second part further comprises two L-shaped wing tips separately connected to the front of the divider which respectively engage the two strip openings with circular openings in the first part, allowing the first part to move back and forth relative to the second part and allowing the first part to bend against the second part about 90 degrees.

6. The container of claim 5, wherein the main carrying case has a lock mechanism to lock the two parts together in place once they are closed to one another.

7. The container of claim 6, wherein the main carrying case has round circular outlets for a clip to hook on to the bottom for carrying.

8. The container of claim 7, wherein the card case comprises a first unit and a second unit connected to one another via a mechanism which allows the first unit to move close to or away from the second unit such that the card case is in a closed or opened position and which allows the first unit to bend against the second unit about 90 degrees such that the cards can be easily accessed.

9. The container of claim 8, wherein the card case further comprises a removable face plate for preventing the content inside card case from falling.

10. The container of claim 9, wherein the card case has a locking mechanism to lock the first and second units in place.

11. The container of claim 10, wherein the second unit comprises a bottom wall, left and right inside walls, left and right outside walls, back wall, and two U-shaped bars connected to the left and right insides and outside walls wherein the flange projects perpendicularly from the side walls towards the inside of the card case.

12. The container of claim 11, wherein the first unit comprises a front wall, a bottom wall, left and right side walls connected to the front wall, and two rectangular bars extending from the right and left side walls, each of which has a rectangular opening and a circle opening at one end of the rectangular opening, the rectangular openings engaging the two U-shaped bars of the second unit, allowing the first unit to move back and forth or bend down relative to the second unit wherein the first unit further comprises a small u shaped top frame that is connected to the front wall and to the inside top part of the first unit.

13. The container of claim 12, wherein the locking mechanism has a lock pin, a C-shaped lock and a backward C-shaped lock.

14. The container of claim 13, wherein the unique removable face plate has a horizontal leg bar to engage a rectangular hollow space located at the back top of the second unit to secure the removable face plate to the connected first and second units wherein there is a horizontal bar with left and right wing tips that can be inserted and secure into the rectangular hollow spaces at the left and right top corners of the second unit.

15. The container of claim 14, wherein the card case further comprises several bars 28 having screw holes in the middle for a clip to hook onto for easy carrying.

16. The container of claim 15, wherein the syringe case comprises:
a top unit having a roof and two parallel bars under the roof; and
a bottom unit having right and left side walls and two long rectangular hollow spaces respectively located inside the right and left side walls for the two bars of the top unit to insert through such that the top unit is removably attached to the bottom unit to close and secure the contents of the syringe case.

17. The container of claim 16, wherein the syringe case further comprises a square shaped bar having a hole in the middle allowing a key ring or a small lock to go through to secure and lock the top and bottom units together when the two combine together.

18. The container of claim 17, wherein the syringe case further comprises scratch guards to prevent the top and bottom units from being scratched by a key ring or a lock.
